(19) United States
(12) Patent Application Publication
(10) Pub. No.: US 2008/0139943 A1
(43) Pub. Date: Jun. 12, 2008

(54) ULTRASONIC WAVE DEVICE

(75) Inventors: Tse-Min Deng, Hsinchu City (TW); Ming-Wei Chang, Taichung County (TW); Te-I Chiu, Hsinjhuang City (TW); Mu-Yueh Chen, Kaoshiung City (TW)

Correspondence Address: Akin Gump LLP - Silicon Valley, 3000 El Camino Real, Two Palo Alto Square, Suite 400, Palo Alto, CA, 94306, US

(73) Assignee: INDUSTRIAL TECHNOLOGY RESEARCH INSTITUTE, Hsinchu (TW)

(21) Appl. No.: 11/567,836

(22) Filed: Dec. 7, 2006

(51) Int. Cl. A61B 8/00

(52) U.S. Cl. 600/459

(57) ABSTRACT

An ultrasonic wave device has a first flexible layer, a second flexible layer, a flexible circuit between the first flexible layer and the second flexible layer, and one or more arrays of capacitive ultrasonic transducer between the first flexible layer and the second flexible layer electrically coupled to the flexible circuit. The array of capacitive ultrasonic transducer is configured to transmit ultrasonic energy through the second flexible layer in response to electrical energy applied via the flexible circuit.

### BACKGROUND OF THE INVENTION

The present invention is generally related to ultrasonic wave technology, and more particularly to an ultrasonic wave device capable of transmitting an ultrasonic wave by direct air-coupling to a test medium.

Phonophoresis is a process whereby an ultrasound energy or ultrasonic wave is used to enhance diffusion of topically applied medicines or cosmetic compounds into the tissues beneath the skin. U.S. Pat. No. 6,322,532 to D'Sa et al. discloses a device for enhancing permeation of a substance through a membrane for purposes of transdermal/transmucosal drug delivery and/or body fluid monitoring. Effectively, the medicines contained within or under the ultrasound gel are pushed by the ultrasonic waves and driven deep below the skin. Also, the ultrasonic wave can stimulate skin tissue and improve blood and lymphatic circulation by massaging effect. U.S. Pat. No. 6,090,054 to Tagishi et al. discloses a hand-held ultrasonic wave device for ultrasonic skin cleaning and skin treatment. The ultrasonic wave generated by the ultrasonic wave vibration elements is applied through a probe of the ultrasonic wave device to the skin.

Generally, in conventional ultrasonic wave devices, piezoelectric materials are selected in the manufacture of the ultrasonic wave vibration elements, such as transducers for the ultrasonic wave cosmetic device. The transducers use piezoelectric material to convert electrical energy into ultrasound. As a result, the temperature generated from the transducers as well as the probe head metal surface that contacts the skin may increase to a level which is too high for human skin. Such an increase in the temperature occurs when the transducers are continuously driven for a long period of time or the ultrasonic wave is concentrated on a fixed point of the skin for a relatively long time. Therefore, the conventional ultrasonic wave devices of this kind may burn or otherwise adversely affect the skin.

Moreover, in the conventional ultrasonic wave devices, a mismatch between acoustic impedance of air and that of a test material may generate enormous resistance for ultrasound propagation. And due to extremely high attenuation of ultrasound by air, the ultrasound transmission in the test material may be usually done by physically contacting or coupling the transducer to the test material using a liquid or gel couplant. Yet, the use of the liquid or gel couplant is sometimes undesirable because it may contaminate or penetrate into the material being tested leading to reduction of mechanical properties or corrosion. Also, the application of certain liquid or gel couplant on skin areas may cause discomfort or even irritation for some users.

### BRIEF SUMMARY OF THE INVENTION

In accordance with the invention, there is provided an ultrasonic wave device that includes a contact patch and a control module in an electrical communication with the contact patch. The contact patch includes a substrate, at least a flexible ultrasonic transducer array arranged on the substrate, at least a circuit arranged adjacent to the flexible ultrasonic transducer array to connect the flexible ultrasonic transducer array to an adjacent flexible ultrasonic transducer array or the control module, and an encapsulating layer disposed over the substrate to encapsulate the flexible ultrasonic transducer array and the circuit.

Also, in accordance with the invention, there is provided an ultrasonic wave device that includes a mask body and an electronic control module in an electrical communication with the mask body. The mask body includes a backing layer, at least a flexible ultrasonic transducer array arranged on the backing layer, at least a flexible circuit arranged adjacent to the flexible ultrasonic transducer array to connect the flexible ultrasonic transducer array to an adjacent flexible ultrasonic transducer array or the electronic control module, and an encapsulating layer disposed over the backing layer for encapsulating the flexible ultrasonic transducer array and the flexible circuit.

Further in accordance with the invention, there is provided an ultrasonic wave device that includes a first flexible layer, a second flexible layer, a flexible circuit between the first flexible layer and the second flexible layer, and at least one array of capacitive ultrasonic transducer between the first flexible layer and the second flexible layer electrically coupled to the flexible circuit. The at least one array of capacitive ultrasonic transducer is capable of transmitting ultrasonic energy through the second flexible layer in response to electrical energy applied via the flexible circuit.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings examples which are presently preferred. It should be understood, however, that the invention is not limited to the precise arrangements and instrumentalities shown.

In the drawings:

FIG. 1 is a schematic side view of one of the ultrasonic transducer elements described in U.S. Pat Publication No. 20040249285A1;

FIG. 2 is a block diagram illustrating a control module powered by a power supply according to one example of the invention;

FIG. 3 is a schematic diagram illustrating a rear view of a mask body and a perspective view of an ultrasonic facial mask according to one example of the invention;

FIG. 4 is a cross-sectional view of the mask body shown in FIG. 3; and

FIG. 5 is a schematic diagram illustrating a rear view of a contact patch and a perspective view of an ultrasonic pad according to another example of the invention.

### DETAILED DESCRIPTION OF THE INVENTION

Examples of the invention may provide an ultrasonic wave device that is made from non-piezoelectric materials and capable of transmitting ultrasonic wave by direct air-coupling to an area. Specifically, examples of the present invention may provide an ultrasonic wave device which generates and transmits an appropriate amount of ultrasonic wave by direct air-coupling to an area. With a plurality of flexible ultrasonic transducers arranged in arrays in the ultrasonic wave device, the ultrasonic wave transmitted by air-coupling penetrates through the area to a certain depth to achieve a variety of phonophoresis effects. In other words, examples of the invention may also provide a flexible ultrasonic wave device capable of enhancing permeability of skin in contact with the flexible ultrasonic wave device.

In accordance with some examples of the invention, the ultrasonic wave device includes a contact patch and a control module in an electrical communication with the contact patch. The contact patch may be assembled from a substrate, at least a flexible ultrasonic transducer array arranged on the substrate, at least a circuit arranged adjacent to the flexible ultrasonic transducer array in such a way as to connect the flexible ultrasonic transducer array to an adjacent flexible ultrasonic transducer array or the control module. And an encapsulating layer may be disposed over the substrate for encapsulating the flexible ultrasonic transducer array and the circuit.

In one example of the invention, the substrate may provide a flexible and biocompatible base for the contact patch of the ultrasonic wave device. For example, the substrate may be made of a biocompatible and flexible material which includes but is not limited to non-woven materials such as non-woven textiles or fabrics; woven materials such as woven textiles or fabrics; and silicone gel material. Therefore, the contact patch that is assembled from the biocompatible and flexible material may be safe to use with a biological tissue. In one example, the flexible material of the substrate provides flexibility to the contact patch when the ultrasonic wave device is used. On the other hand, the substrate may be made of a non-flexible material which includes but is not limited to polycarbonate and Teflon if a firm base for the contact patch is desired for certain applications.

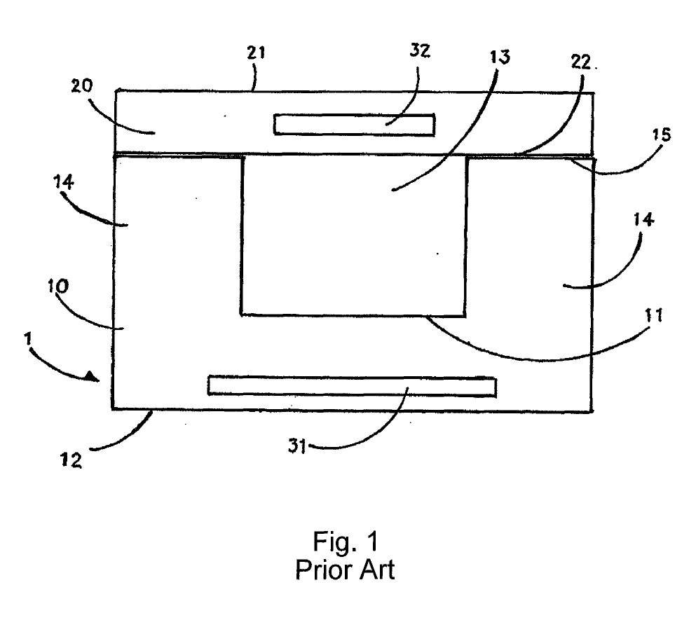

According to another example of the invention, a number of flexible ultrasonic transducers are provided and arranged in arrays on the substrate at required areas. An example of the flexible ultrasonic transducer may be found in U.S. Pat Publication No. 20040249285A1, filed previously by Deng et al., one of the inventors of the present invention. The flexible ultrasonic transducer is formed by combining a plurality of ultrasonic transducer elements on an extended base. FIG. 1 is a reproduction of FIG. 1 of U.S. Pat Publication No. 20040249285A1. Referring to FIG. 1, each of the ultrasonic transducer elements 1 may include a membrane 20, a first electrode 31, and a second electrode 32. The base 10, which may be a structure that extends horizontally, may have an upper side 11 and a lower side 12. In one example, the horizontal base 10 and a lateral support structure 14 form as a single body having a depression 13 cut into the upper side 11 of the base 10, with the support 14 set on the outer edge of the base 10 thereof. The support 14 has at its upper end a top side 15. The membrane 20 has an outer (top) side 21 and an inner (bottom) side 22, which is placed on the top side 15 of the support 14. The first electrode 31 is inserted within the base 10 between the upper and lower sides 11 and 12 of the base 10. The second electrode 32 is inserted within the membrane 20 between the outer and inner sides 21 and 22 of the membrane 20. The arrangement shown in FIG. 1 may provide certain distance between the first and second electrodes 31 and 32 and may provide a relatively thick insulating layer to allow large vibration amplitude of the membrane. As a result, a relatively large amount of electrical energy may be stored between the first and second electrodes 31 and 32 for driving the membrane 20 and for being converted to mechanical energy for vibrations. Skilled persons in the art will understand that the present invention is not limited to the particular example illustrated. Other capacitive ultrasonic transducers may be used.

The flexible ultrasonic transducer arrays may be assembled from a plurality of flexible ultrasonic transducers by a physical method or a chemical method at areas of the substrate requiring the ultrasonic energy. On a surface of the substrate, the flexible ultrasonic transducer arrays are coupled to each other and to the control module via the circuits, which may be arranged adjacent to or in the proximity of the flexible ultrasonic transducer arrays. The flexible ultrasonic transducer arrays may include flexible, capacitive, micro-machined ultrasonic transducer arrays or any flexible, air-coupled, ultrasonic transducer arrays. The encapsulating layer may then be disposed or placed over the substrate to encapsulate the flexible ultrasonic transducer arrays and the circuits. In some examples, the encapsulating layer may be made of a biocompatible material which includes but is not limited to parylene, SU-8 and other polymer materials.

In one example, the circuit that couples the flexible ultrasonic transducer arrays to each other and to the control module may be a flexible circuit which includes metal leads or connectors. The flexible circuit may be made from materials which include but are not limited to polyimide and kepton. The circuit may also be made from non-flexible material for certain applications, such as for applications where a firm base with no or limited flexibility of the contact patch is required. The flexible circuit may couple the flexible ultrasonic transducer arrays to the control module via a connector. As described in some examples of the invention, the control module may be an electrical device coupled to the contact patch to provide a driving power and switch function for the ultrasonic wave device. As an example, the control module may be coupled to the contact patch permanently. In another example, the control module may be coupled to the contact patch using a detachable connector which can be separated from the contact patch and the control module. The detachable design may provide flexibility in packing and unpacking the ultrasonic wave device.

In some other examples, the ultrasonic wave device may also include a fastening means for affixing the contact patch to an object or a desired area of an object. The fastening means may be attached to the edge or center portion of the contact patch as long as the fastening means helps to keep the contact patch in place and affixed to the desired area. Alternatively, the fastening means may be made as a detachable part which may be separated from the contact patch when needed. For example, the fastening means no longer needs to associate with the contact patch when the contact patch is frequently moved around for providing ultrasound waves at various areas or when the contact patch needs to be expanded to cover an extended area. The fastening means may include at least one of a fastening tape, a fastening strap, a fastening band, a fastening belt and a fastening chain.

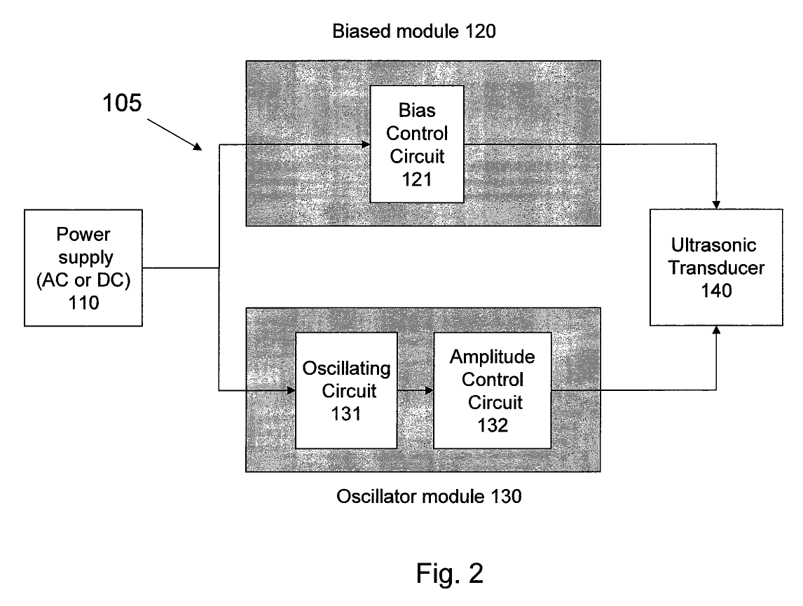

According to an example of the present invention, the ultrasonic wave device may be powered by a power supply 110 via a control module 105. As shown in FIG. 2, the control module 105 includes a bias module 120 and an oscillator module 130. In operation, the power supply 110 provides voltage signals to the bias module 120 and the oscillator module 130. The bias module 120 has a bias control circuit 121. In the bias module 120, the voltage signals received by the bias control circuit 121 are output to an ultrasonic transducer 140. The oscillator module 130 has an oscillating circuit 131 coupled to an amplitude control circuit 132. So, the voltage signals received by the oscillating circuit 131 are output to the amplitude control circuit 132. The amplitude control circuit 132 then outputs the signals to the ultrasonic transducer 140. Therefore, the outputs from the bias module 120 and the oscillator module 130 are fed to the ultrasonic transducer 140 to generate the ultrasonic wave.

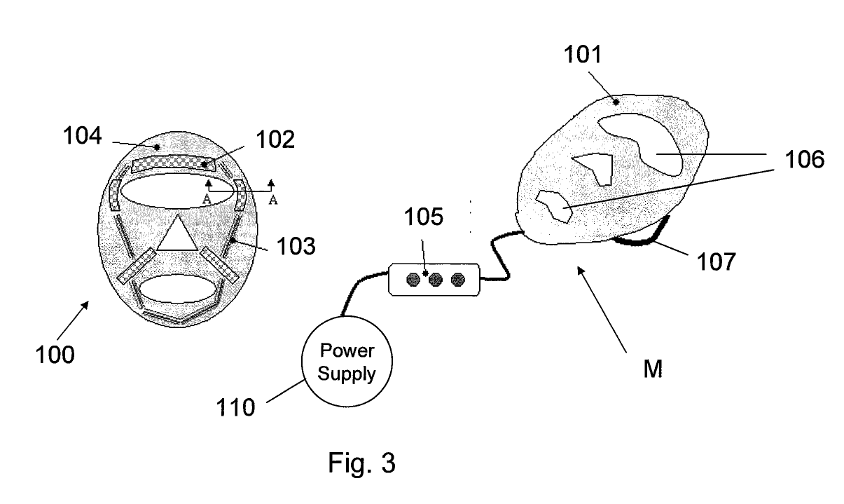

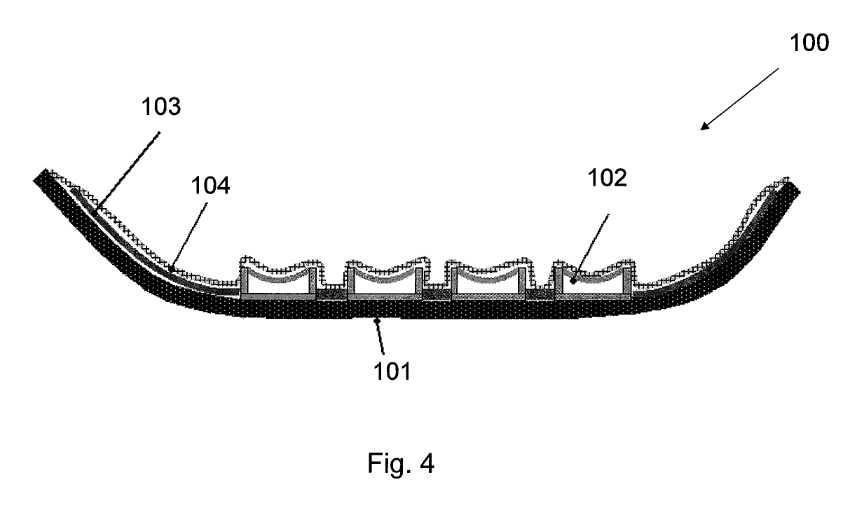

One example of the invention is described with reference to FIGS. 3 and 4. As shown in FIG. 3, the ultrasonic wave device may be configured as an ultrasonic facial mask 1. The ultrasonic facial mask M includes a mask body 100 and an electronic control module 105 in electrical communication with the mask body 100. The electronic control module 105 may have a plug to connect to the power supply 110. Referring to FIG. 4, the mask body 100 may be assembled from a flexible backing layer 101. A flexible ultrasonic transducer array 102 may be arranged on the flexible backing layer 101, and a flexible circuit 103 may be arranged adjacent to the flexible ultrasonic transducer array 102 for coupling the flexible ultrasonic transducer array 102 to an adjacent flexible ultrasonic transducer array 102. And an encapsulating layer 104 may be formed over the backing layer 101 for encapsulating the flexible ultrasonic transducer array 102 and the flexible circuit 103. Since the mask body 100 is assembled from flexible materials, the ultrasonic facial mask M fabricated accordingly may conform to user's face contour. Referring to FIG. 3, one or more openings 106 may be provided by the mask body 100 to allow an user to see, breath, drink or even eat through the at least one opening 106 when the ultrasonic facial mask M is worn. In other examples, a plurality of openings 106 may be provided at areas of the mask body 100 corresponding to the user's eyes, nose and mouth. And the mask body 100 also has a fastening strap 107 for affixing the mask body 100 to a desired facial area of the user.

In operation, when the user wears the ultrasonic facial mask M on his/her face, the control module 105 coupled to the mask body 100 may be switched ON to drive the flexible ultrasonic transducer arrays 102. The flexible ultrasonic transducer arrays 102 then generate ultrasonic waves at a frequency range of approximately 0.5 to 3 MHz. Specifically, the ultrasonic waves such as a pulse wave having a power of approximately 0.1 to 0.5 W/cm² and a continuous wave having a power of approximately 0.5 to 1.5 W/cm² may be applied. Hence, with the ultrasonic waves generated by the flexible ultrasonic transducer arrays 102, the ultrasonic facial mask M may provide massages to the user's facial skin.

According to other examples of the invention, a liquid, colloidal or gel medium may be applied on the user's face before wearing the ultrasonic facial mask to further enhance the transmission of ultrasonic wave. Therefore, in certain applications of the invention, the ultrasonic facial mask may be used in combination with other cosmetic products or skin care items, such as cosmetic facial mask. As the cosmetic products are evenly applied on the user's face, they can penetrate the facial skin of the user more easily due to enhanced skin permeability and absorption by phonophoresis.

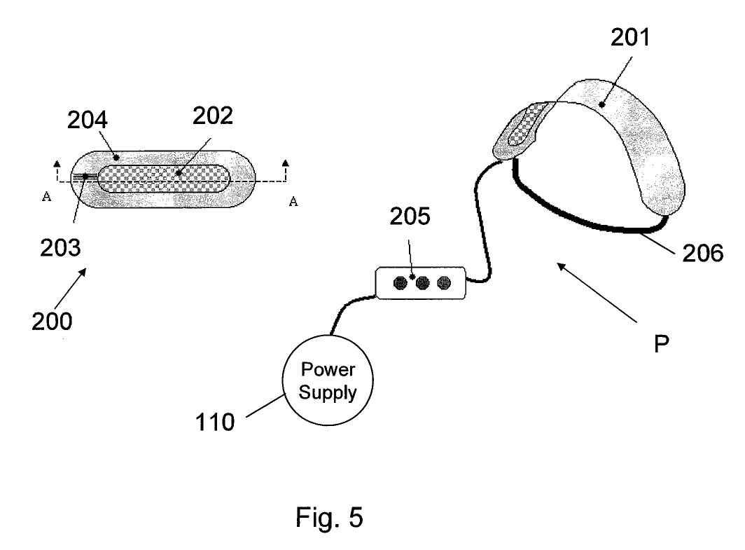

In another example, the ultrasonic wave device may be configured as an ultrasonic pad P illustrated in FIG. 5. The ultrasonic pad P may be used on human body, for example the limbs or specific body parts to relieve tiredness of the body. Referring to FIG. 5, the ultrasonic pad P includes a contact patch 200 and a control module 205 in electrical communication with the contact patch 200. The control module 205 may have a plug to connect to the power supply 110. The contact patch may be assembled from a substrate 201, at least a flexible ultrasonic transducer array 202 arranged on the substrate 201, at least a circuit 203 arranged adjacent to the flexible ultrasonic transducer array 202. And an encapsulating layer 204 is formed over the substrate 201 for encapsulating the flexible ultrasonic transducer array 202 and the circuit 203. The contact patch 200 also has a fastening strap 206 for affixing the contact patch 200 to the desired body area. In operation, the ultrasonic transducer array 202 may be driven by the control module 205 to generate the ultrasonic wave at a frequency capable of achieving desired phonophoresis or massage effects to the user.

The ultrasonic wave device applicable to the invention shall not be limited to those configurations described above. The ultrasonic wave device may also be designed to have different shapes, sizes or configurations, including but not limited to cuffs, bands, hoods, masks and gloves depending on the user's needs. Therefore, the present invention may also provide an ultrasonic wave device having a modified structure. For example, a device may include a first flexible layer, a second flexible layer, a flexible circuit between the first flexible layer and the second flexible layer, and one or more arrays of capacitive ultrasonic transducer between the first flexible layer and the second flexible layer electrically coupled to the flexible circuit. The array or arrays of capacitive ultrasonic transducer may be capable of transmitting ultrasonic energy through the second flexible layer in response to electrical energy applied via the flexible circuit.

In view of the medical and cosmetic benefits provided, it is understood by one having ordinary skills in the art to possibly incorporate, build or couple the ultrasonic wave device to currently available medical instruments, devices or tools, so as to achieve results and benefits associated with the ultrasonic wave transmission. For example, the ultrasonic wave provided by the ultrasonic wave device may help to relieve body fatigue or tiredness. Thus, the ultrasonic wave device may be designed as a massage machine and used in conjunction with other devices.

Moreover, the ultrasonic wave transmission provided by the ultrasonic wave device also enhances blood circulation and provides warming effects. So, it is encompassed by the scope of the invention that the ultrasonic wave device may be embedded or built as a part of the body wears or body carriers to provide beneficial effects at any location. And it is generally accepted that the ultrasonic wave device of the invention may also be applicable to other fields in need of ultrasound transmission by direct air-coupling to the test material without having problems associated with the conventional ultrasonic wave devices.

Other examples of the invention will be apparent to those skilled in the art from consideration of the specification and practice of the invention disclosed herein. It is intended that the specification and examples be considered as exemplary only, with a true scope and spirit of the invention being indicated by the following claims.

It will be appreciated by those skilled in the art that changes could be made to the examples described above without departing from the broad inventive concept thereof. It is understood, therefore, that this invention is not limited to the particular examples disclosed, but it is intended to cover modifications within the spirit and scope of the present invention as defined by the appended claims.

I/we claim:

1. An ultrasonic wave device comprising
a mask body, the mast body comprising:
a backing layer,
at least a flexible ultrasonic transducer array arranged on the backing layer,
at least a flexible circuit arranged adjacent to the flexible ultrasonic transducer array for coupling the flexible ultrasonic transducer array to an adjacent flexible ultrasonic transducer array or the electronic control module, and
an encapsulating layer over the backing layer for encapsulating the flexible ultrasonic transducer array and the flexible circuit; and
an electronic control module in electrical communication with the mask body.

2. The device according to claim 1, wherein the mask body further comprises a fastening means for affixing the mask body to a facial area in need of the facial mask.

3. The device according to claim 1, wherein the mask body provided at least one opening.

4. The device according to claim 1, wherein the backing layer comprises a flexible material.

5. The device according to claim 1, wherein the encapsulating layer comprises a biocompatible material.

6. The device according to claim 1, wherein the flexible circuit includes a flexible metal lead.

7. The device according to claim 1, wherein the electronic control module is coupled to the mask body via a permanent, conductive wire.

8. The device according to claim 7, wherein the electronic control module is arranged on the mask body.

9. The device according to claim 1, wherein the electronic control module is coupled to the mask body via a detachable connector.

10. An ultrasonic wave device comprising a contact patch and a control module in electrical communication with the contact patch, wherein the contact patch comprises:
a substrate;
at least a flexible ultrasonic transducer array arranged on the substrate;
at least a circuit arranged adjacent to the flexible ultrasonic transducer array for coupled the flexible ultrasonic transducer array to an adjacent flexible ultrasonic transducer array or the control module; and
an encapsulating layer over the substrate for encapsulating the flexible ultrasonic transducer array and the circuit.

11. The device according to claim 10, wherein the contact patch further comprises a fastening means for affixing the contact patch to a desired area.

12. The device according to claim 10, wherein the substrate comprises a flexible material.

13. The device according to claim 10, wherein the flexible ultrasonic transducer array includes a flexible, capacitive, ultrasonic transducer array.

14. The device according to claim 10, wherein the encapsulating layer comprises a biocompatible material.

15. The device according to claim 10, wherein the one circuit includes a flexible metal lead.

16. The device according to claim 10, wherein the electronic control module is coupled to the contact patch via a permanent, conductive wire.

17. The device according to claim 16, wherein the electronic control module is arranged on the contact patch.

18. The device according to claim 10, wherein the electronic control module is coupled to the contact patch via a detachable connector.

19. The device according to claim 10, the contact patch provides at least one opening.

20. An ultrasonic wave device, comprising:
a first flexible layer;
a second flexible layer;
a flexible circuit between the first flexible layer and the second flexible layer; and
at least one array of capacitive, ultrasonic transducer between the first flexible layer and the second flexible layer electrically coupled to the flexible circuit, the at least one array of capacitive ultrasonic transducer being configured to transmit ultrasonic energy through the second flexible layer in response to electrical energy applied via the flexible circuit.
